(19) United States
(12) Patent Application Publication
(10) Pub. No.: US 2018/0028066 A1
(43) Pub. Date: Feb. 1, 2018

(54) OBJECT INFORMATION ACQUIRING APPARATUS AND METHOD FOR DISPLAYING IMAGE RELATING TO OBJECT

(71) Applicant: CANON KABUSHIKI KAISHA, Tokyo (JP)

(72) Inventors: Moemi Urano, Tokyo (JP); Kenichi Nagae, Yokohama-shi (JP)

(73) Assignee: Canon Kabushiki Kaisha, Tokyo (JP)

(21) Appl. No.: 15/548,181

(22) PCT Filed: Mar. 3, 2016
(86) PCT No.: PCT/JP2016/057481

§ 371 Date: Aug. 2, 2017

(30) Foreign Application Priority Data
Mar. 4, 2015 (JP) 2015-042732
Mar. 1, 2016 (JP) 2016-039289

(51) Int. Cl. A61B 5/00; G01S 7/52

(52) U.S. Cl. CPC A61B 5/0095; A61B 5/489; A61B 5/7278; A61B 5/7425; G01S 7/52038; A61B 2576/02

(57) ABSTRACT

An object information acquiring apparatus including: an extraction processing unit that extracts signal components of mutually different first and second frequency bands from an electric signal based on an acoustic wave propagating from an object due to irradiation of the object with light; an image signal generating unit that generates a first image signal based on the signal component of the first frequency band extracted by the extraction processing unit, a second image signal based on the signal component of the second frequency band extracted by the extraction processing unit, and a third image signal based on the electric signal; and a weighting processing unit that performs weighting of a signal intensity of the third image signal on the basis of signal intensities of the first and second image signals.

### TECHNICAL FIELD

The present invention relates to an object information acquiring apparatus and a method for displaying an image relating to an object.

### BACKGROUND ART

Imaging apparatus which are object information acquiring apparatus using X-rays, ultrasound waves and MRI (nuclear magnetic resonance method) have been widely used in the medical field. Meanwhile, optical imaging apparatus that obtain information on the inside of a living body, which is object information, by irradiating the living body, which is the object, with light from a light source, such as a laser, and causing the light to propagate in the living body have been actively researched in the medical field. Photoacoustic imaging technique is one of such optical imaging techniques.

The photoacoustic imaging is described hereinbelow. Thus, an object is irradiated with pulsed light generated from a light source. Then, an acoustic wave (also referred to as “photoacoustic wave”) generated by the living body tissue, which is the light absorbing body that absorbs the energy of light propagating and diffusing in the object, is detected. The obtained signal is analyzed and information relating to optical property values (a type of object information) of the object interior is visualized.

Incidentally, when a living body is irradiated with light that is absorbed by blood, imaging of blood vessels can be performed. For example, it has been suggested to divide the detected signal of a photoacoustic wave generated inside an object by the irradiation of the object with light into a low-frequency component and a high-frequency component, and generate a photoacoustic image in which an image constituted by the low-frequency component is corrected using the image constituted by the high-frequency component (PTL 1). A technique has also been suggested by which a photoacoustic image is objected to spatial frequency processing (PTL 2).

### CITATION LIST

### Patent Literature

PTL 1: Japanese Patent Application Publication No. 2013-233386

PTL 2: Japanese Patent Application Publication No. 2013-176414

### SUMMARY OF INVENTION

### Technical Problem

However, a living body has blood vessels of a variety of thicknesses. Where photoacoustic imaging is performed on such a structure, thick objects tend to be bright and thin objects tend to be dark. As a result, visibility of thin blood vessels is inhibited.

Further, in PTL 1, since the image constituted by the low-frequency component is corrected on the basis of the image constituted by the high-frequency component, the visibility of thin blood vessels cannot be expected to be improved. In PTL 2, since the images are objected to spatial frequency processing, image reconstruction is difficult to perform accurately with respect to a signal of a round columnar shape which is typical for a blood vessel structure.

With the foregoing in view, it is an objective of the present invention to provide an object information acquiring apparatus and a method for displaying an image relating to an object, the apparatus and method making it possible to enhance selectively a structure.

### Solution to Problem

To attain the abovementioned objective, the present invention provides the following configuration. Thus, provided is an object information acquiring apparatus including: an extraction processing unit that extracts signal components of mutually different first and second frequency bands from an electric signal based on an acoustic wave propagating from an object due to irradiation of the object with light; an image signal generating unit that generates a first image signal based on the signal component of the first frequency band extracted by the extraction processing unit, a second image signal based on the signal component of the second frequency band extracted by the extraction processing unit, and a third image signal based on the electric signal; and a weighting processing unit that performs weighting of a signal intensity of the third image signal on the basis of signal intensities of the first and second image signals.

The present invention also provides the following configuration. Thus, provided is a object information acquiring apparatus including: an extraction processing unit that extracts a signal component of a first frequency band from an electric signal based on an acoustic wave propagating from an object due to irradiation of the object with light; an image signal generating unit that generates a first image signal based on the signal component of the first frequency band extracted by the extraction processing unit, a second image signal obtained on the basis of the electric signal, without using the extraction processing unit, and a third image signal based on the electric signal; and a weighting processing unit that performs weighting of a signal intensity of the third image signal on the basis of signal intensities of the first and second image signals.

The present invention also provides the following configuration. Thus, provided is a method for displaying an image relating to an object, including: a step of displaying a first photoacoustic image relating to a group of blood vessels in the object; and a step of performing image processing on a first blood vessel and a second blood vessel differing in thickness from the first blood vessel, which are contained in the group of blood vessels, the image processing to be performed on the first blood vessel being different from that performed on the second blood vessel whereby a second photoacoustic image is formed, wherein the image processing is performed such that a visibility of the first blood vessel with respect to the second blood vessel in the first photoacoustic image is different from a visibility of the first blood vessel with respect to the second blood vessel in the second photoacoustic image.

### Advantageous Effects of Invention

As indicated hereinabove, the present invention provides an object information acquiring apparatus and a method for displaying an image relating to an object, the apparatus and method making it possible to enhance selectively a structure.

Further features of the present invention will become apparent from the following description of exemplary embodiments with reference to the attached drawings.

### BRIEF DESCRIPTION OF DRAWINGS

FIG. 1 is a block diagram illustrating Example 1 of the object information acquiring apparatus of the present invention.

FIG. 2 is a flowchart illustrating the functions of the object information acquiring apparatus of Example 1.

FIG. 3 is a relationship diagram of the frequency of the digital signal of Example 1 and the brightness value of image data.

FIG. 4 illustrates the reconstructed image data of Example 1.

FIG. 5 is a flowchart illustrating Example 2 of the object information acquiring apparatus of the present invention.

FIG. 6 illustrates the frequency band extracted by the filter circuit of Example 2.

FIGS. 7A to 7C illustrate the reconstructed image data of Example 2 and the displayed image based on the enhanced image signal.

FIG. 8 illustrates the functions of the filter circuit of Example 3 of the object information acquiring apparatus of the present invention.

FIG. 9 illustrates the functions of the filter circuit of Example 4 of the object information acquiring apparatus of the present invention.

FIG. 10 illustrates the functions of the filter circuit of Example 5 of the object information acquiring apparatus of the present invention.

### DESCRIPTION OF EMBODIMENTS

The embodiments of the present invention will be explained hereinbelow in detail with reference to the appended drawing. In principle, like constituent elements are assigned hereinbelow with like reference numerals, and the explanation thereof is omitted. The specific computational formulas and computational procedure described hereinbelow are intended to be changed, as appropriate, according to the configuration and conditions of the device using the present invention, and the scope of the invention is not intended to be limited to the description below.

The object information acquiring apparatus of the present invention is inclusive of devices using a photoacoustic effect in which an acoustic wave generated in an object (for example, breast, face, palm, etc.) due to irradiation of the object with light (electromagnetic wave), such as near-infrared radiation, is received and object information is acquired as image data.

In the case of apparatus using a photoacoustic effect, the object information which is to be acquired indicates the generation source distribution of acoustic waves generated by light irradiation, the initial sound pressure distribution inside the object, the light energy absorption density distribution or absorption coefficient distribution derived from the initial sound pressure distribution, and concentration distribution of substances constituting the tissue. The concentration distribution of substances is, for example, an oxygen saturation degree distribution, total hemoglobin concentration distribution, and oxidation-reduction hemoglobin concentration distribution.

Further, the property information, which is object information on a plurality of positions, may be acquired as a two-dimensional or three-dimensional property distribution. The property distribution can be generated as image data illustrating property information on the inside of the object. The acoustic wave, as referred to in the present invention, is typically an ultrasound wave and is inclusive of elastic waves called sound waves and ultrasound waves. An acoustic wave generated by the photoacoustic effect is referred to herein as a photoacoustic wave or photoultrasound wave. The acoustic wave detector (for example, a probe) receives the acoustic waves generated in the object.

### Example 1

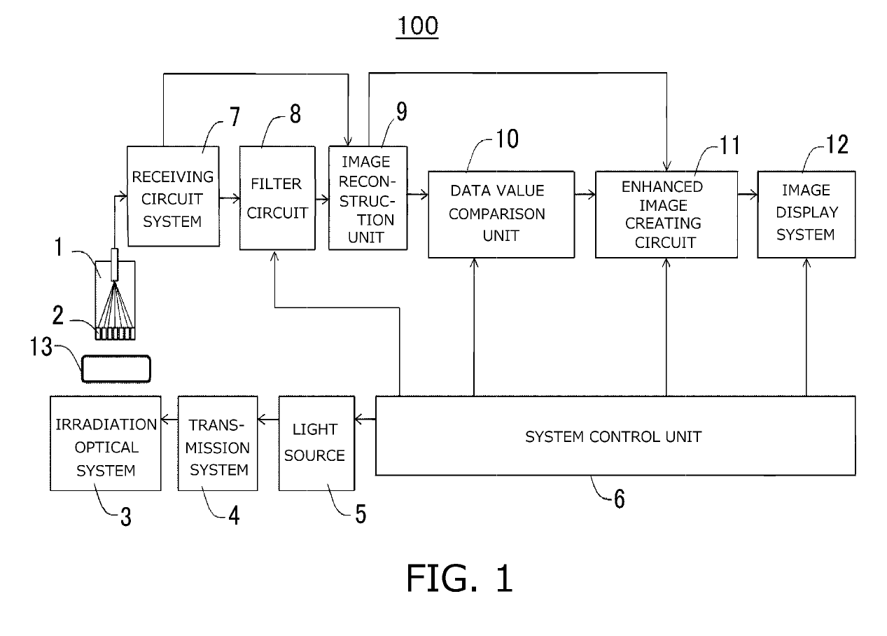

FIG. 1 is a block diagram illustrating Example 1 of the object information acquiring apparatus according to an embodiment of the present invention. The object information acquiring apparatus 100 (referred to hereinbelow as “apparatus 100”) of Example 1 has a probe 1 (corresponds to the receiving unit), acoustic wave detection elements 2, an irradiation optical system 3 (corresponds to the irradiation unit), a transmission system 4, and a light source 5. The apparatus 100 also has a system control unit 6, a receiving circuit system 7, a filter circuit 8 (corresponds to the extraction processing unit), an image reconstruction unit 9 (corresponds to the image signal generating unit), a data value comparison unit 10 (data value difference detection unit), an enhanced image signal creating circuit 11 (corresponds to the weighting processing unit), and an image display system 12 (corresponds to the display unit).

The light source 5 emits pulsed light on the basis of a control signal from the system control unit 6. The irradiation optical system 3 shapes the pulsed light generated from the light source 5 into the desired light shape and irradiates an object 13 with the shaped light. The light generated by the light source 5 may be pulsed light with a pulse width of about 10 nsec to 100 nsec. Such light enables efficient generation of photoacoustic waves. The light source 5 is preferably a high-output laser, but is not limited thereto, and may be a light-emitting diode or a flash lamp rather than a laser. Various lasers such as a solid-state laser, a gas laser, a dye laser, and a semiconductor laser can be used in the light source 5. The wavelength of the light generated by the light source 5 is preferably such that enables light propagation into the object 13. For example, when the object 13 is a living body, the wavelength may be 500 nm (inclusive) to 1200 nm (inclusive). Further, such a configuration is not limiting, and the laser used in the light source 5 may be a high-output laser with a continuously variable wavelength, for example, a Nd:YAG-excited Ti:sa laser or an alexandrite laser. Further, the light source 5 may include a plurality of single-wavelength lasers having different wavelengths.

The transmission system 4 transmits the pulsed light from the light source 5 to the irradiation optical system 3. As indicated hereinabove, a light absorbing body (angiogenesis, cancer, etc.) in the object 13 generates photoacoustic waves by absorbing the energy of the light by which the object 13 is irradiated. The transmission system 4 may be configured, for example, by a multi-joint arm in which a plurality of hollow waveguide tubes are connected by joints enclosing mirrors which is configured to enable the propagation of light inside the waveguide tube. Alternatively, the propagation of light in the space can be ensured by optical elements such as mirrors and lenses. The transmission system 4 may be also configured by band fibers.

The probe 1 is configured by arranging a plurality of acoustic wave detection elements 2. The acoustic wave detection elements 2 receive the photoacoustic wave propagating from the object 13 and convert the photoacoustic wave into an electric signal (received signal). The acoustic wave detection elements 2 using a piezoelectric effect, light resonance, or changes in electrostatic capacity may be used. Such options are, however, not limiting, and the acoustic wave detection elements of any type may be used, provided that acoustic waves can be received. The acoustic wave detection elements 2 may be configured by arranging a plurality, for example, of piezo elements one-dimensionally, two-dimensionally, or sterically. By using acoustic wave detection elements 2 arranged multidimensionally, such as a plurality of piezo elements (any elements capable of receiving acoustic waves), it is possible to receive acoustic waves at a plurality of positions at the same time. Therefore, the measurement time can be reduced.

When the plurality of the acoustic wave detection elements 2 is arranged sterically in the probe 1, the arrangement thereof may be such that the direction with the highest reception sensitivity of each acoustic wave detection element 2 is towards (concentrated on) a predetermined region in the object 13. For example, the plurality of the acoustic wave detection elements 2 may be arranged along a substantially semicircular surface. The acoustic wave detection elements 2 also transmit the electric signals converted thereby from the output terminal of the probe 1 to the receiving circuit system 7 of the later stage.

The receiving circuit system 7 implements the sampling processing or amplification processing on the received signals outputted from the probe 1, converts them into digital signals (received signals after digital conversion), and transmits the digital signals to the filter circuit 8. Further, when the below-described correction (weighting) of image intensity is performed on the reconstructed image based on the digital signals which have not been objected to filter processing, the digital signals from the receiving circuit system 7 are also directly inputted to the image reconstruction unit 9. The receiving circuit system 7 is configured, for example, of a signal amplifier such as an operational amplifier or an analog/digital converter (ADC).

In the filter circuit 8, the digital signal inputted from the receiving circuit system 7 is objected to filter processing in the frequency band designated by the system control unit 6, and a signal formed from signal components in the predetermined frequency bands configured by the filter processing is transmitted to the image reconstruction unit 9. The filter processing may be performed by cutting off the frequencies outside the predetermined frequency band designated by the system control unit 6, or by attenuating the signal components outside the predetermined frequency band to extract the signal components in the predetermined frequency band. In the filter circuit 8, in this case, the signal components in the predetermined frequency band may be extracted such that the signal component is gradually reduced with the increasing distance from the central frequency of the predetermined frequency band. When the extraction in the filter circuit 8 is performed by reducing the signal components outside the predetermined frequency band, the extraction may be performed such that the signal components at a greater distance from the central frequency of the predetermined frequency band decay to a greater extent. Further, the frequency band of the signal components extracted in the filter circuit 8 may be determined, as appropriate, according to the thickness of the observation object (blood vessel, or the like). In this case, the frequency of the photoacoustic wave generated in the living body may be determined with consideration for the dependence on the thickness of the light absorbing body. Further, when the light absorbing body is a sphere, the frequency of the photoacoustic wave may be determined by using the generation of the photoacoustic wave in an N-type shape. Thus, the frequency of the photoacoustic wave may be determined by taking an inverse value of the time width t of the N-type shape. The time width t may be determined by dividing the diameter d of the light absorbing body by the sound velocity c with the CPU of the apparatus 100.

The image reconstruction unit 9 performs image reconstruction processing by using signal data transmitted from the filter circuit 8. The image reconstruction, as referred to herein, is for example, the processing of calculating the initial sound pressure distribution p(r) of the photoacoustic waves inside the object 13 by using Filtered Black Projection (FBP). The Filtered Black Projection (FBP) is an image reconstruction method using the distribution presented by Formula (1) below.

$$\begin{matrix} [ Math . 1 ] & \\ p ( r ) = - \frac{1}{2 π} {\int }_{{s}_{0}}^{ } \int {\frac{{dS}_{0}}{{r}_{0}^{2}} [ t \frac{\partial {p}_{d} ( {r}_{0} , t )}{\partial t} + 2 {p}_{d} ( {r}_{0} , t ) ]}_{t = \vert r - {r}_{0} \vert / c} & Formula ( 1 )\end{matrix}$$

In Formula (1) above, dS₀ is the size of the detector, S₀ is the size of the aperture used for the reconstruction, p_{d}(r, t) is the signal received by each acoustic wave detection element, t is the reception time, and r₀ is the position of each acoustic wave detection element.

The image reconstruction unit 9 transmits the reconstruction data generated by performing the image reconstruction processing to a data value comparison unit 10 or the enhanced image signal creating circuit 11. The image reconstruction unit 9 performs the image reconstruction based on the unfiltered digital signals from the receiving circuit system 7, and the below-described enhanced image signal creating circuit 11 performs the intensity weighting with respect to the obtained reconstructed image. In this case, unfiltered image data are generated by performing the image reconstruction processing also on the digital signal directly inputted from the receiving circuit system 7, and the generated image data are transmitted to the enhanced image creating circuit 11. The image data in this case are objected to intensity weighting by the enhanced image creating circuit 11. The image reconstruction unit 9 may be configured, for example, by a CPU (including a multicore CPU), FPGA, work station, or hardware.

In the data value comparison unit 10, the intensity difference value distribution information, which is a value based on the difference in intensity (brightness value, contrast value, etc.) between two image data is calculated by using two image data generated by the image reconstruction processing. However, such a configuration is not limiting, and a value based on the difference in intensity between three or more image data may be calculated in the data value comparison unit 10. The data value comparison unit 10 may be configured, for example, by a CPU (including a multicore CPU), FPGA, work station, or hardware.

The enhanced image signal creating circuit 11 performs weighting (correction) of the intensity of one of the two image data by using the intensity difference value distribution information. The enhanced image signal creating circuit 11 outputs the image display data which have been enhanced in intensity as a result of the intensity weighting to the image display system 12. The image display system 12 serves as a user interface and displays the inputted image display data as visible images. The enhanced image signal creating circuit 11 may be configured, for example, by a CPU (including a multicore CPU), FPGA, work station, or hardware.

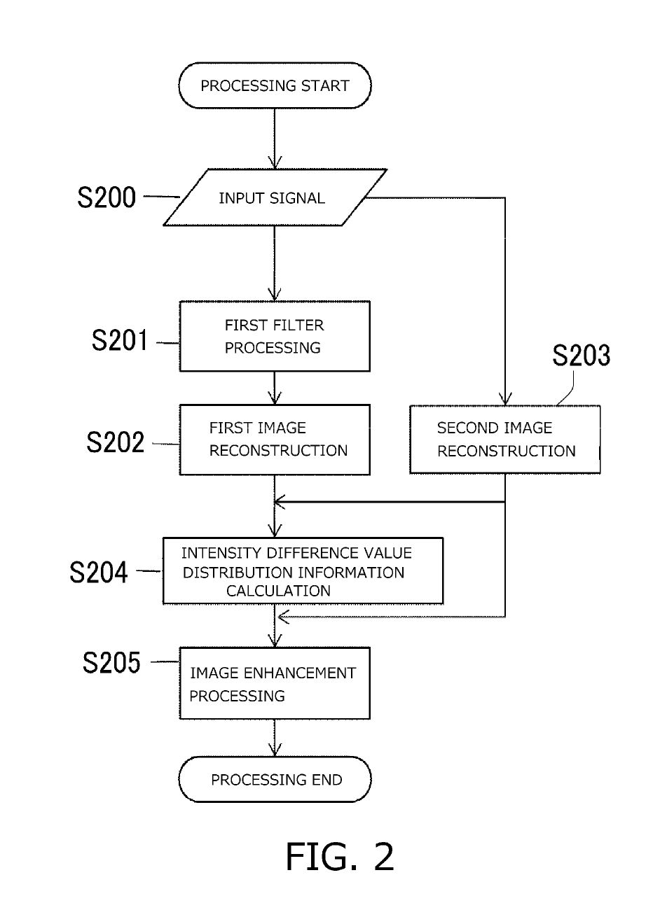

FIG. 2 is a flowchart illustrating the functions of the filter circuit 8 to the enhanced image signal creating circuit 11, which are depicted in FIG. 1, in Example 1 of the present invention. The flow is started by the digital conversion with the receiving circuit system 7 and formation of a digital signal. In step S200, the digital signal from the receiving circuit system 7, is inputted as an input signal into the filter circuit 8 and the image reconstruction unit 9, and the flow advances to step S201 and step S203.

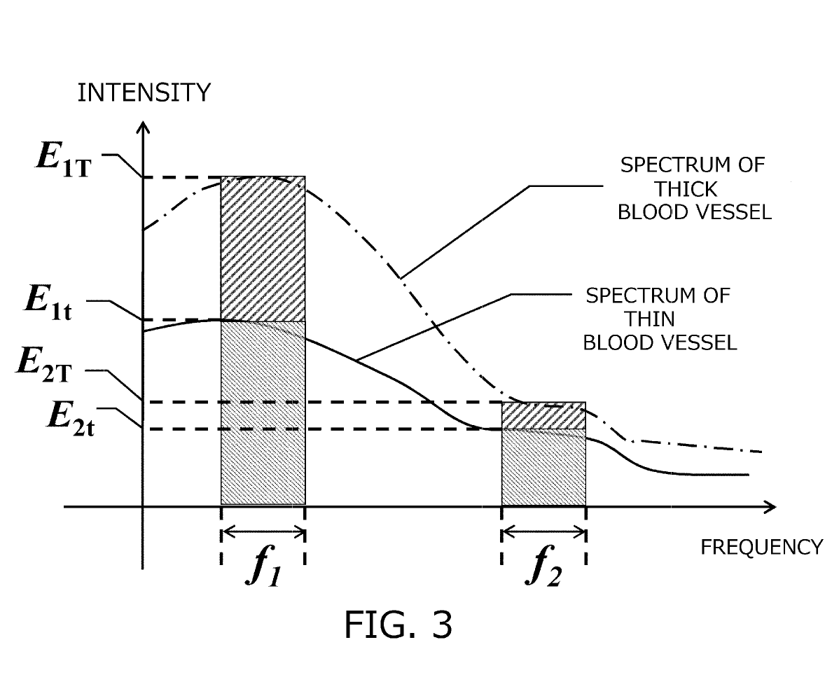

In step S201, a frequency band to be extracted by the filter circuit 8 is designated by the user through a user interface (monitor, or the like) which is not depicted in the figure. At this time, the user may input the frequency band manually with a keyboard, or the like, on a monitor, or may select from a plurality of choices. When selecting from a plurality of choices, a frequency band in which fine blood vessels are predominant (for example, a band f2 in the below-described FIG. 3) and a frequency band in which thick blood vessels are predominant (for example, a band f1 in the below-described FIG. 3) are determined by a test, or the like, in advance, and stored in a memory, etc. The set value of the frequency band which is wished to be extracted is then read, as appropriate, according to the user's selection from the memory. Where the frequency band indication is inputted by the user to the system control unit 6, as indicated hereinabove, a frequency band determination signal corresponding to this indication is inputted from the system control unit 6 to the filter circuit 8. A time constant in the filter circuit 8 is determined on the basis of this input, and the frequency band which is to be extracted is determined. The extraction of signals is performed such that the intensity of the digital signal from the receiving circuit system 7 is gradually reduced following the increase in distance from the central frequency of the determined frequency band in the frequency direction. The signal components of the frequency band are extracted, and the flow advances to step S202. However, such a procedure is not limiting, and the filter processing may be performed by reducing the signal components outside the determined frequency band.

By using the filter of the frequency band inputted by the user, it is possible to display the thickness of the structure which is to be enhanced. It is also possible to refer to the table representing the relationship between the specific frequency and the thickness of the structure which is to be enhanced, this table having been stored in advance in a memory, or the like. Another option is to calculate the brightness values obtained by filtering the frequency band designated by the user with respect to the photoacoustic waves generated from various thicknesses and display the calculation results as changes in the brightness value in relation to the thickness.

Further, as a result of designating the thickness of the blood vessel which is wished to be enhanced by the user, instead of directly designating the frequency band of the filter, the system control unit 6 may automatically set the frequency band of the filter suitable for the thickness. This can be realized when the table representing the relationship between the specific frequency and the thickness of the structure which is to be enhanced is stored in advance in a memory, or the like.

In step S202, the signal formed by filter processing is inputted to the image reconstruction unit 9, and the image reconstruction unit 9 performs the image reconstruction processing on the basis of the inputted signal, thereby generating the first image data. The processing flow then advances to step S204. In step S203, the second image data are generated by performing the image reconstruction processing with the image reconstruction unit 9 on the basis of the digital signal sent from the receiving circuit system 7. The processing flow then advances to step S204 and step S205.

In step S204, the below-described division processing is performed with the data value comparison unit 10 after the processing of step S202 and step S203 has been executed. Thus, the brightness value at the coordinate (1, m, n) of the first image data is divided by the brightness value at the same coordinates (l, m, n) of the second image data. The division processing is performed with respect to all of the coordinates of the first image data (second image data). As a result of performing the division processing, a value based on the difference in signal intensity (in this case, the brightness value) between the first and second image data is calculated. The value based on the difference in brightness values, which are signal intensities, is calculated for each coordinate of the first image data (second image data). The processing flow then advances to step S205. The data set of the values based on the difference in signal intensities which has been calculated for each coordinate of the first image data may be taken as “intensity difference value distribution information”. For convenience of explanation, “the intensity difference value distribution information” refers to the entire data set and also the constituent elements at each coordinate of the data set are also referred to, one by one, as “the intensity difference value distribution information”.

Such an approach is not limiting, and the first and second image data may be one-dimensional or two-dimensional image data. Furthermore, it is possible to acquire a value obtained by adding an offset, etc., to the abovementioned division processing result for all coordinates and define the data sets as “the intensity difference value distribution information”.

In step S205, the intensity of the second image data is weighted by using the intensity difference value distribution information after the processing of step S204 and step S203 has been executed. Performed in this case is the processing of multiplying the brightness values at each coordinate of the second image data by the intensity difference value distribution information (the constituent elements) at the same coordinates. This multiplication processing is performed with respect to all of the coordinates of the second image data. The enhanced image signal is formed by thus enhancing the brightness at each coordinate of the second image data, and the processing flow is ended. Such an approach is, however, not limiting, and the intensity of the first image data may be weighted by using the intensity difference value distribution information. It is thus possible to form the enhanced image signal by enhancing the brightness at each coordinate of the first image data. In the explanation hereinabove, the intensity of image data is taken as the brightness value, but such an approach is not limiting, and the contrast value, etc., of image data may be taken as the intensity of image data.

In such a case, less filtering in the filter circuit 8 is required and it is sufficient to extract one frequency band. Therefore, the processing volume is reduced and the calculation time and cost can be reduced.

The principle of selective enhancement of blood vessels is explained hereinbelow.

FIG. 3 illustrates the relationship between the frequency of the digital signal outputted from the receiving circuit system 7 and the brightness value of image data in Example 1 of the present invention. The filter circuit 8 extracts the signal component of a specific frequency band among the signals from the receiving circuit system 7 and outputs the extracted signal component. The image reconstruction unit 9 forms image data by image reconstruction on the basis of the extracted signal. The brightness value of the image data is determined on the basis of the intensity of the signal component included in the specific frequency band. In FIG. 3, typical spectra of a thick blood vessel and a thin blood vessel are shown to explain this determination. The dot-dash line in FIG. 3 shows the spectrum of the thick blood vessel, and the solid line in FIG. 3 shows the spectrum of the thin blood vessel. FIG. 3 also shows an approximate energy amount E_{1T} included in the low-frequency band f1 of the thick blood vessel and an approximate energy amount E_{2T} included in the high-frequency band f2. Also shown in FIG. 3 are an approximate energy amount E₁ₜ included in the low-frequency band f1 of the thin blood vessel and an approximate energy amount E₂ₜ included in the high-frequency band f2. In the case illustrated by FIG. 3, the following relationship is valid between the ratio (E_{2T}/E_{1T}) of the energy amounts included in the frequency bands with respect to the spectrum of the thick blood vessel and the ratio (E₂ₜ/E₁ₜ) of the energy amounts included in the frequency bands with respect to the spectrum of the thin blood vessel.

(E_{2T}/E_{1T})<(E₂ₜ/E₁ₜ) Formula (2)

Thus, the ratio (E_{2T}/E_{1T}) of the energy amounts included in the frequency bands with respect to the spectrum of the thick blood vessel is less than the ratio (E₂ₜ/E₁ₜ) of the energy amounts included in the frequency bands with respect to the spectrum of the thin blood vessel. The ratio of the energy amount contained in the frequency band f1 and the energy amount contained in the frequency band f2 in the digital signal from the receiving circuit system 7 differs according to the blood vessel thickness. By using this fact, it is possible to determine a value based on the difference in intensity necessary for weighting the intensity, even without restrictively using the frequency band corresponding to the blood vessel thickness.

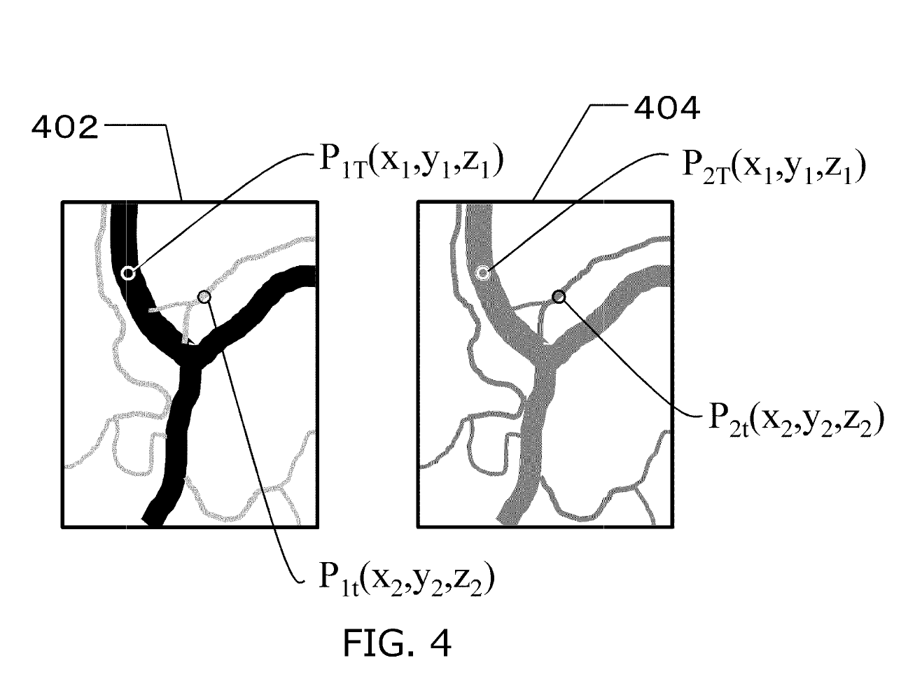

FIG. 4 shows the reconstructed image data of Example 1. A blood vessel image is included in the figure. FIG. 4 shows low-frequency band image data 402 and high-frequency band image data 404. Represented in FIG. 4 are the displayed images which are based on the low-frequency band image data 402 and high-frequency band image data 404 in the case in which the display on the display device is performed on the basis of the respective data, but those displayed images are also referred to as image data for convenience of explanation. The low-frequency band image data 402 are formed by performing image reconstruction of a digital signal formed by extracting the signal component of the frequency band f1 in FIG. 3 with respect to the digital signal inputted from the receiving circuit system 7. The high-frequency band image data 404 is formed by performing image reconstruction of a digital signal formed by extracting the signal component of the frequency band f2 in FIG. 3. In the high-frequency band image data 404 formed by the reconstruction in the image reconstruction unit 9, the brightness value corresponding to the thin blood vessel becomes comparatively high. By contrast, in the low-frequency band image data 402 formed by the reconstruction in the image reconstruction unit 9, the brightness value corresponding to the thick blood vessel becomes comparatively high.

In the enhanced image signal creating circuit 11, the brightness value allocated to the coordinates (l, m, n) of the high-frequency band image data 404 is divided by the brightness value allocated to the coordinates (l, m, n) of the low-frequency band image data 402. As a result, enhanced difference value distribution information α may be obtained. In the enhanced image signal creating circuit 11, the brightness value P(x1, y1, z1) at the coordinates (x1, y1, z1) of the high-frequency band image data 404 is divided by the brightness value Q(x1, y1, z1) at the coordinates (x1, y1, z1) of the low-frequency band image data 402. In the enhanced image signal creating circuit 11, as a result of such division, it is possible to determine the enhanced difference value distribution information α(x1, y1, z1) at the coordinates (x1, y1, z1) of the high (low)-frequency band image data 404 (402). In the enhanced image signal creating circuit 11, the enhanced difference value distribution information α at each coordinate may be determined by performing the above-described division processing with respect to each coordinate (xk, yk, zk) (k=1, 2, . . . , n). Thus, in the enhanced image signal creating circuit 11, each enhanced difference value distribution information α(xk, yk, zk), such as represented by the formulas below, may be determined. Further, in the enhanced image signal creating circuit 11, the enhanced difference value distribution information α at each coordinate may be determined only for some, rather than all, of the coordinates.

α(x1,y1,z1)=P(x1,y1,z1)/Q(x1,y1,z1)

α(x2,y2,z2)=P(x2,y2,z2)/Q(x2,y2,z2) . . . ,

α(xk,yk,zk)=P(xk,yk,zk)/Q(xk,yk,zk) . . . ,

α(xn,yn,zn)=P(xn,yn,zn)/Q(xn,yn,zn) Formula (3)

In the enhanced image signal creating circuit 11, the high-frequency band image data 404 (the intensity of the high-frequency band image data 404 at each coordinate is represented hereinbelow by “P₀”) is multiplied by each enhanced difference value distribution information α(xk, yk, zk) calculated on the basis of Formula 3 above. Thus, the enhanced image signal creating circuit 11 may generate an enhanced image signal (the intensity of the enhanced image signal at each coordinate is represented hereinbelow by “Pₒᵤₜ”).

For example, the enhanced image signal creating circuit 11 may generate the Pₒᵤₜ by computational processing based on Formula 4 below. The intensity Pₒᵤₜ of the enhanced image signal at each coordinate is generated hereinabove by multiplying the intensity P₀ of the high-frequency band image data 404 at each coordinate by each enhanced difference value distribution information α(xk, yk, zk). Such a procedure is, however, not limiting, and the enhanced image signal may be also generated by multiplying the image data formed by direct image reconstruction by each enhanced difference value distribution information α(xk, yk, zk), without extracting the specific frequency band of the digital signal from the receiving circuit system 7, as explained with reference to FIG. 2. The “image data formed by direct image reconstruction”, as referred to herein, are for example the image data generated in step S203 in FIG. 2.

Pₒᵤₜ(x1,y1,z1)=P₀(x1,y1,z1)×α(x1,y1,z1)

Pₒᵤₜ(x2,y2,z2)=P₀(x2,y2,z2)×α(x2,y2,z2)

Pₒᵤₜ(xk,yk,zk)=P₀(xk,yk,zk)×α(xk,yk,zk)

Pₒᵤₜ(xn,yn,zn)=P₀(xn,yn,zn)×α(xn,yn,zn) Formula (4)

The enhanced image signal creating circuit 11 may perform the intensity weighting for each intensity P₀ at each coordinate of the high-frequency band image data 404 on the basis of Formula 4, as indicated hereinabove. When Formulas 3 and 4 above are used, the value of enhanced difference value distribution information α at coordinates corresponding to the position of the thick blood vessel is less than that of the respective enhanced difference value distribution information α at the coordinates corresponding to the position of the thin blood vessel. Therefore, in the enhanced image signal creating circuit 11, the brightness at the coordinate corresponding to the thick blood vessel in the image data is decreased and the brightness at the coordinate corresponding to the thin blood vessel is increased by multiplying the intensity P₀ at the coordinate by the enhanced difference value distribution information α. In the enhanced image signal creating circuit 11, the enhanced image signal may be generated and this enhanced image signal may be sent to the image display system 12 by performing such brightness weighting processing (multiplication processing) with respect to the intensity P₀ at all coordinates of the image data 404. In this case, in the enhanced image signal, the brightness of the thin blood vessel is enhanced and the brightness of the thick blood vessel is reduced. However, such an approach is not limiting, and in the enhanced image signal creating circuit 11, the enhanced image signal may be generated by performing such brightness weighting processing (multiplication processing) with respect to the intensity P₀ at only some coordinates of the image data 404.

The image display system 12 displays an image in which the visibility of the thin blood vessel is increased on the basis of the enhanced image signal.

As indicated hereinabove, the apparatus 100 can generate the enhanced image signal, in which a random thickness is enhanced, by using the difference in intensity between the spectra of the thick structure (thick blood vessel, etc.) and thin structure (thin blood vessel, etc.). In the present embodiment, the “random thickness”, as referred to herein, is the thickness of the thin blood vessel.

Thus, by using the present invention, it is possible to generate an image signal, in which the structure of a random thickness is enhanced, with respect to structures of various thicknesses, display the image signal, and provide object information (in this case, the blood vessel image in which the brightness of the thin blood vessel is enhanced) with increased visibility.

### Example 2

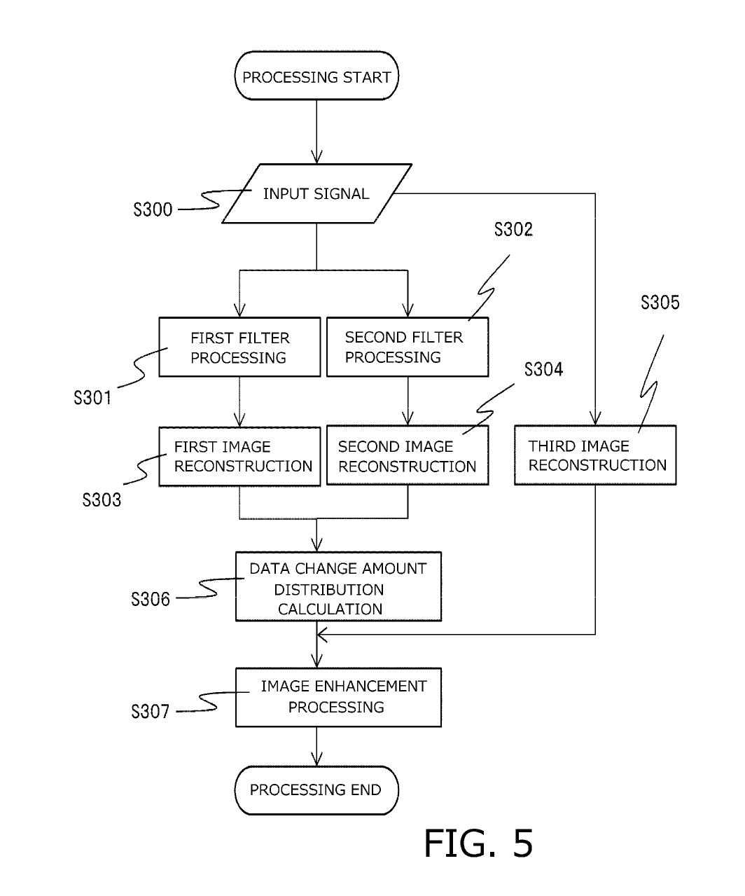

FIG. 5 is a flowchart illustrating the functions of components, from the filter circuit 8 to the enhanced image signal creating circuit 11, in the object information acquiring apparatus of Example 2. The flow starts, in the same manner as described hereinabove, when the receiving circuit system 7 forms a digital signal by performing the digital conversion of the electric signal from the probe 1. In step S300, the digital signal from the receiving circuit system 7 is inputted to the filter circuit 8 and the image reconstruction unit 9, and the flow advances to step S301, step S302, and step S305.

In step S301, the frequency band information on the high frequency side (for example, a designation signal designating 2 MHz to 6 MHz) designated by the user is inputted from the system control unit 6 to the filter circuit 8. Then, the signal component of 2 MHz to 6 MHz, which is the designated specific frequency band, this signal component being part of the signal component of the digital signal inputted from the receiving circuit system 7, is extracted by the filter circuit 8 on the basis of the frequency band information. The processing flow then advances to step S303.

Meanwhile in step S302, the frequency band information on the low frequency side (for example, a designation signal designating 0 MHz to 2 MHz) designated by the user is inputted from the system control unit 6 to the filter circuit 8. Then, the signal component of 0 MHz to 2 MHz, which is the designated specific frequency band, this signal component being the component of the digital signal inputted from the receiving circuit system 7, is extracted by the filter circuit 8 on the basis of the frequency band information. The processing flow then advances to step S304. In this case, the configuration may be used in which the frequency band which is to be extracted or the time constant of the filter circuit can be designated by the user and the designation result may be the above-mentioned designation signal. Further, when the filter circuit 8 is configured to have a plurality of filters, the desired filter may be selected, as appropriate, by the user from the plurality of filters. In this case, the configuration may be used such that the selected filter can be verified by the user with an operation screen (not depicted in the figure). Further, the frequency band which is to be extracted by the filter circuit 8 is at least two frequency bands which are entirely separated from each other as will be described hereinbelow. Those at least two frequency bands are not mutually overlapping frequency bands. Such a configuration is, however, not limiting, and the object information acquiring apparatus of the present example can be used in a similar manner when extracting three or more frequency bands which are not mutually overlapping frequency bands. Further, the range of the frequency band which is to be extracted by the filter may be determined in advance or may be designated each time by the user.

In step S303, the signal of the extracted frequency band on the low frequency side is inputted to the image reconstruction unit 9, and image reconstruction is performed by the image reconstruction unit 9 on the basis of this signal. The processing flow then advances to step S306. Meanwhile, in step S304, the signal of the extracted frequency band on the high frequency side is likewise inputted to the image reconstruction unit 9, and image reconstruction is performed by the image reconstruction unit 9 on the basis of this signal. The processing flow then advances to step S306. The image reconstruction used herein uses the above-mentioned FBP.

After the processing of step S303 and step S304 has ended, the data after the two reconstruction operations are inputted to the data value comparison unit in step S306. The brightness value at each coordinate of the image data of the frequency band on the high frequency side is divided by the brightness value at each coordinate of the image data of the frequency band on the low frequency side. The division is performed between the same coordinates. The intensity difference value distribution information α is calculated for each coordinate, and the processing flow then advances to step S307. Meanwhile, in step S305, the digital signal (input signal) from the receiving circuit system 7 before the filter processing is inputted to the image reconstruction unit 9. This digital signal is objected to reconstruction processing in the image reconstruction unit 9, non-filtered image data are formed, and the processing flow advances to step S307. The non-filtered image data, as referred to herein, indicate image data that have been formed by image reconstruction without performing filter processing.

In this procedure, before the division between the same coordinates is performed, spatial smoothing processing may be implemented with respect to the reconstructed data obtained in step S303 and step S304. By performing such smoothing processing, it is possible to suppress a noise component contained in the reconstructed image, thereby making it possible to improve the accuracy of the obtained intensity difference value distribution information α.

Further, the division processing may be also performed after adding a very small amount to the reconstructed data obtained in step S303 and step S304, thereby making it possible to suppress an error caused in the intensity difference value distribution information α by division by 0.

Furthermore, the intensity difference value distribution information α may be objected to smoothing processing or median processing, and such processing can suppress an error included in the intensity difference value distribution information α, thereby making it possible to enhance the structure of a specific thickness with better accuracy.

After the processing of step S306 and step S305 has ended, the non-filtered image data are multiplied by the calculated intensity difference value distribution information α in step S307 to generate the enhanced image signal. The processing flow is thus ended. A visible image is then formed by the image display system 12 on the basis of the enhanced image signal, and the formed image is displayed on the operation screen of a monitor which is the user interface.

In the present example, the non-filtered image data formed by image reconstruction processing of the input signal before the filter processing are multiplied by the intensity difference value distribution information α in order to generate the enhanced image signal. However, such a configuration is not limiting, and the same effect can be also obtained by multiplying the image data formed by filter processing in the frequency band on the high frequency side and the image reconstruction processing by the intensity difference value distribution information α. In such a case, the visibility of the thick blood vessel is decreased by the filter processing in a high frequency band, but the visibility of the thin blood vessel is easily improved. Further, by multiplying the image data formed by filter processing in the frequency band on the low frequency side and the image reconstruction processing by the intensity difference value distribution information α, the brightness of the thicker blood vessel is increased. As a result, the visibility of the thick blood vessel is further improved.

Further, the intensity difference value distribution information α may use an exponential or logarithmic function. As a result, the objects which are too thin can be eliminated and a more natural image can be obtained. Further, the operator may interactively change the coefficients of the intensity difference value distribution information α. As a result, it is possible to enhance a blood vessel of a thickness which the operator wishes to obtain.

In the present embodiment, the case is explained in which FBP is used for image reconstruction, but an image reconstruction method using a Hilbert transform may be also used for image reconstruction.

An image reconstruction method using the Hilbert transform in the present invention includes repeating for each position of interest a step of transforming the signal received by each element into complex data by the Hilbert transform, a step of picking up complex data from the Hilbert-transformed received signal of each element with consideration for the delay of the reception time which has been calculated from the position of interest where image reconstruction is to be performed, the distance to each element, and the sound velocity, and a step of summing up the picked-up complex data and calculating the absolute value thereof. With such a method, the image of the region of interest is eventually obtained.

This method makes it possible to visualize the energy of the photoacoustic wave generated from each position of interest. Since the energy is visualized, no negative values are produced as a result of image reconstruction.

Therefore, in the division processing performed in step S306, the operation of dividing by zero or a negative value can be suppressed. As a result, the intensity difference value distribution information α can be calculated with better stability. Therefore, an image which is unlikely to cause an uncomfortable feeling can be obtained with the enhanced image signal calculated in step S307.

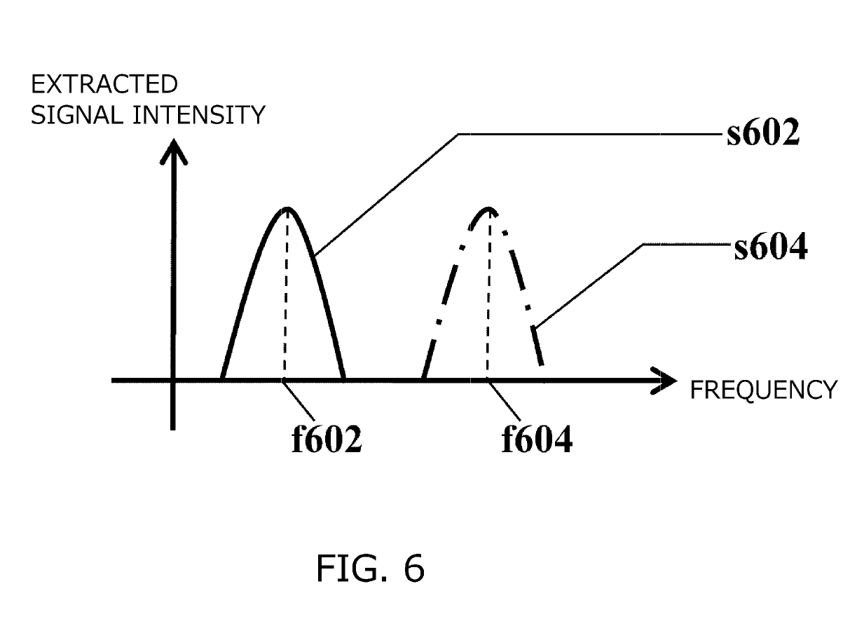

FIG. 6 shows the frequency band extracted by the filter circuit of Example 2. In FIG. 6, the frequency (Hz) is plotted against the abscissa, and the signal intensity which is to be extracted is plotted against the ordinate. In FIG. 6, the filter circuit 8 extracts two signals, namely, a high-frequency band extracted signal s604 and a low-frequency band extracted signal s602. In the case illustrated by FIG. 6, the filter circuit 8 performs the extraction such that the frequency bands of those two signals do not overlap. The central frequencies of the high-frequency band extracted signal s604 and the low-frequency band extracted signal s602 are a frequency f604 and a frequency f602 which are average values of the frequencies included in the band which is wished to be extracted and at which the respective signal intensities are substantially at a maximum. The signals s602, s604 attenuate symmetrically in the positive and negative directions of the frequency axis, the respective central frequencies f602, f604 substantially being the centers.

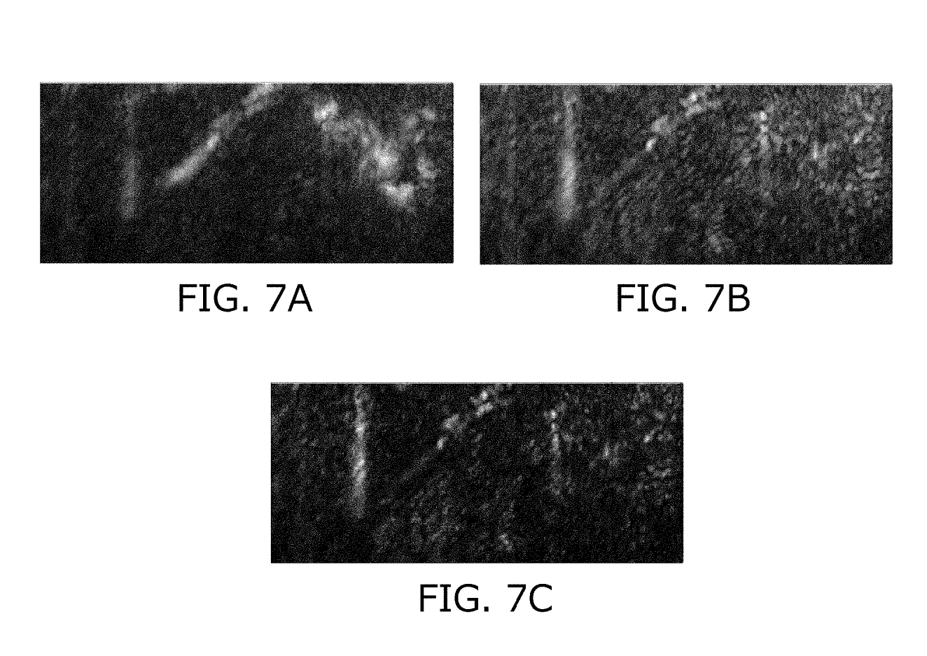

FIG. 7 shows the displayed images based on the image data after image reconstruction and enhanced image signal in Example 2. FIG. 7A is the displayed image formed by filter processing and image reconstruction in a frequency band (0 MHz to 2 MHz) on a low-frequency side. FIG. 7B is the displayed image formed by filter processing and image reconstruction in a frequency band (2 MHz to 6 MHz) on a high-frequency side. FIG. 7C is the displayed image formed on the basis of the enhanced image signal. In the image state depicted in FIG. 7C, a thin blood vessel is selectively enhances and visibility is improved with respect to those of the images after the filter processing, which are depicted in FIGS. 7A and 7B. It can be seen from FIG. 7C that in the filter circuit 8, the extraction is performed for the low-frequency band and high-frequency band, and the intensity is weighted on the basis of the extracted signals in the processing block of a later stage, thereby producing an image with a high S/N ratio. The object information acquiring apparatus of the present example is particularly effective when the thickness of blood vessels is 1 mm or less.

Further, in the present embodiment, both the reconstructed data obtained in step S303 and step S304 and the non-filtered image data are used for computations in the entire region of interest, but masking performed with a SNR (Signal Noise Ratio) in respective data may be also added.

For example, it is possible to extract only a region having a predetermined or higher SNR in each of the reconstructed data obtained in step S303 and the reconstructed data obtained in step S304, and determine the intensity difference value distribution information α by dividing the extraction results. With such processing, a highly accurate intensity difference value distribution information α can be calculated and a structure of a random thickness can be selectively enhanced with better accuracy. Additional enhancement of the structure of a random thickness can be also performed by including, for example, 0 or a number less than that of the region where the intensity difference value distribution information α has been calculated in the region where the intensity difference value distribution information α is not calculated.

Further, by enhancing the region having a predetermined or higher SNR in the non-filtered image data and then performing the image enhancement processing by using the intensity difference value distribution information α, it is possible to enhance further the structure and to enhance the object of a random thickness and also improve visibility. The same effect can be also obtained by enhancing the region having a predetermined or higher SNR in the non-filtered image data after performing the image enhancement processing with respect to the non-filtered image data.

### Example 3

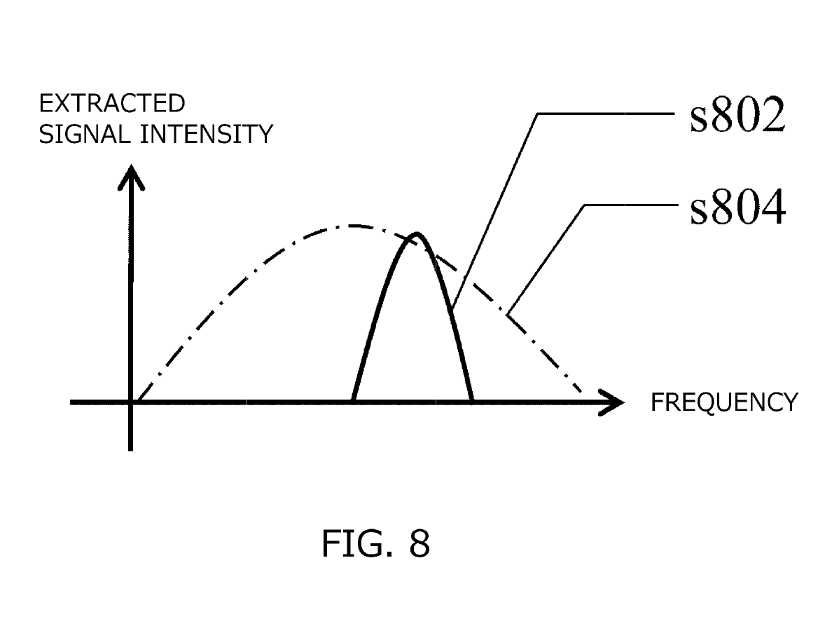

FIG. 8 illustrates the functions of the filter circuit 8 of Example 3 of the object information acquiring apparatus according to the embodiment of the present invention. For convenience, components different from those of Examples 1 and 2 are explained below. The frequency band extracted by reducing part of the input signal in the filter circuit 8 of the present example is different from those in Examples 1 and 2.

In FIG. 8, the filter circuit 8 outputs signals s802, s804 of two frequency bands. In this case, the filter circuit 8 performs the extraction such that the frequency band of one signal s804, from among the two frequency bands, includes the frequency band of the other signal s802. In the filter circuit 8, one frequency band may be processed to be the entire frequency band of the digital signal inputted from the receiving circuit system 7. In this case, the filter circuit 8 may input the digital signal from the receiving circuit system 7 to either of two input terminals. The filter circuit 8 may then output, as is, that is, without any reduction, the digital signal inputted to one input terminal. The filter circuit 8 may also output the digital signal, which has been inputted to the other input terminal, after performing the extraction such that the signal is gradually reduced from the central frequency of the extraction object so as to include the signal in the frequency band of the signal outputted without filtering. The processing of the subsequent stages may be the same as in Example 1 or 2.

In such a case, when a blood vessel image is formed on the basis of the signals of the overlapping frequency bands, this blood vessel image is based on the image data that have been individually objected to image reconstruction on the basis of the signals of the two frequency bands. However, where the signal included in the common band is due to a common blood vessel, it is relatively unlikely that the divisor becomes 0 and the solution diverges in the course of the above-described division processing. In this way, in a case where a blood vessel image based on the same blood vessel is present in the overlapping portion, this being the case in which one of the two frequencies bands encompasses the other, a stable effect of the filter can be obtained.

### Example 4

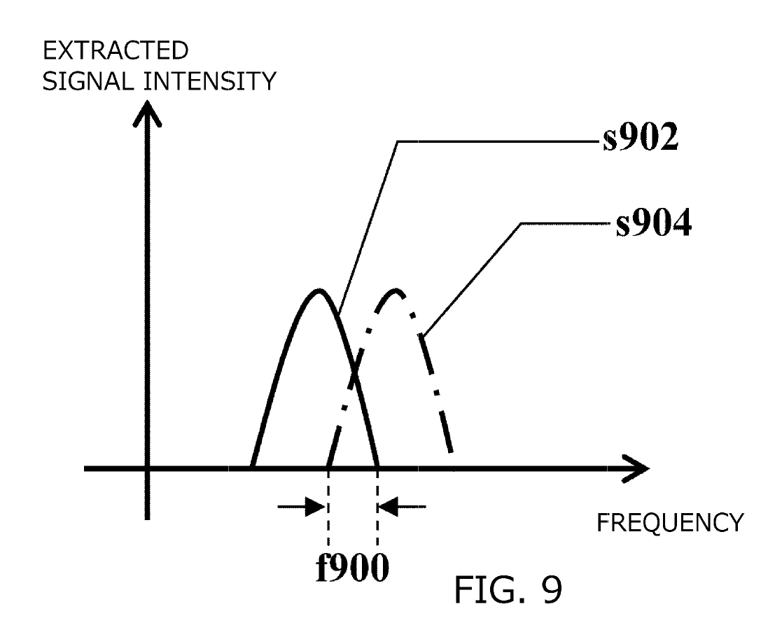

FIG. 9 illustrates the functions of the filter circuit 8 of Example 4 of the object information acquiring apparatus according to the embodiment of the present invention. The constituent elements same as those in Examples 1, 2, and 3 are assigned with same reference numerals, and the explanation thereof is herein omitted. For convenience, components different from those of Examples 1, 2, and 3 are explained below. The frequency band extracted by reducing the input signal in the filter circuit 8 of the present example is different from those in Examples 1, 2, and 3.

In FIG. 9, the filter circuit 8 outputs signals of two frequency bands. In this case, the filter circuit 8 performs the extraction such that part of the frequency band of a signal s902 includes part of the frequency band of a signal s904. Thus, the frequency bands of the signal s902 and the signal s904 each have a common frequency band f900. In this case the filter circuit 8 may input the digital signal inputted from the receiving circuit system 7 to each of two input terminals. The filter circuit 8 may then reduce the digital signal, which has been inputted to one input terminal, outside the first frequency band and output the resultant signal. The filter circuit 8 may also reduce the digital signal, which has been inputted to the other input terminal, outside the second frequency band such that partially overlaps the first frequency band, and output the resultant signal. The processing of the subsequent stages may be the same as in Example 1 or 2. The reduction processing may involve gradual reduction along the positive-negative direction of the frequency axis from the central frequency of each of the first and second frequency bands.

Considering such a case in the same manner as described hereinabove, when a blood vessel image due to a common blood vessel is formed on the basis of the signals of the overlapping frequency bands, it is relatively unlikely that the divisor becomes 0 and the solution diverges in the course of the above-described division processing. In this way, in a case where the extracted signal after the filter processing based on the same blood vessel is present in the overlapping portion, this being the case in which a filter is used with partial overlapping in two different frequency bands, a highly accurate image can be obtained.

### Example 5

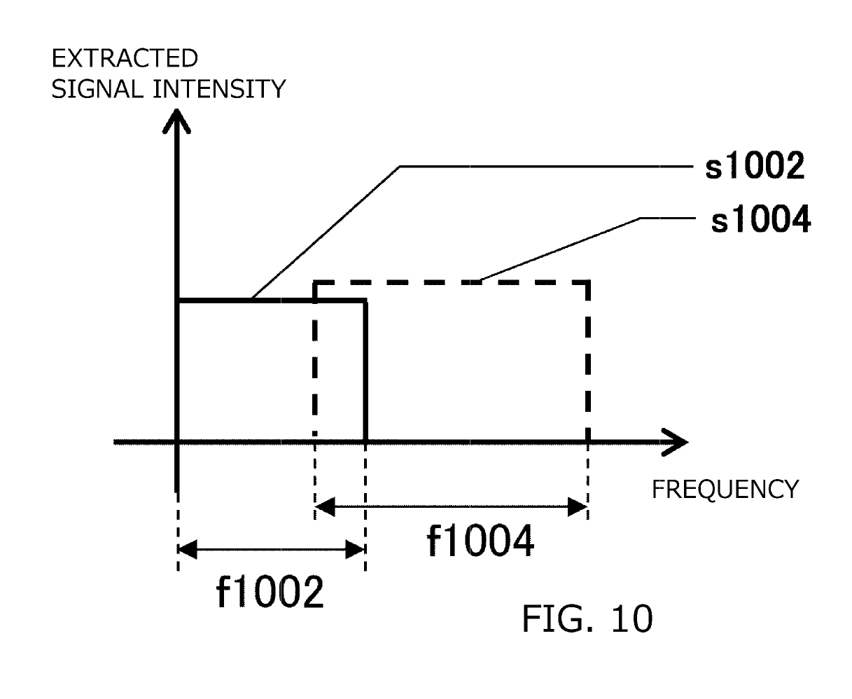

FIG. 10 illustrates the functions of the filter circuit 8 of Example 5 of the object information acquiring apparatus according to the embodiment of the present invention. For convenience, components different from those of Examples 1, 2, 3, and 4 are explained below. The frequency band extracted by cutting off the input signal in the filter circuit 8 of the present example is different from those in Examples 1, 2, 3, and 4.

In FIG. 10, the filter circuit 8 outputs a signal s1002 and a signal s1004 of two frequency bands. In this case, the filter circuit 8 may perform the extraction such that part of one frequency band of the two frequency bands includes part of the other frequency band of the two frequency bands. In such a case, the filter circuit 8 may input the digital signal inputted from the receiving circuit system 7 to each of two input terminals. The filter circuit 8 may then cut off, with a high-pass filter, the frequencies outside a high-frequency band side f1004 of the digital signal inputted to one input terminal and output the signal s1004. The filter circuit 8 may also cut off, with a low-pass filter, the frequencies outside a low-frequency band side f1002 of the digital signal inputted to the other input terminal and output the signal s1002.

As depicted in FIG. 10, the signal intensity of the extracted signals of the frequency bands f1002, f1004 extracted by the low-pass filter and high-pass filter, respectively, may be made uniform. Thus, the low-pass filter of the filter circuit 8 may perform the extraction with a uniform signal intensity over the entire frequency band which needs to be extracted. The same is true with respect to the high-pass filter of the filter circuit 8. The processing of the subsequent stages may be the same as in Example 1 or 2.

In this way, where a high-pass filter is used for the filter on the high frequency side in the two different frequency bands, it is possible to obtain an image in which ringing is suppressed due to the filter processing in a wider band.

### Other Examples

In the above-described embodiment, image data of two types are inputted for data change amount distribution calculations in step S306, but image data of three types, which have been obtained as a result of image reconstruction using signals obtained with filters of three types, may be also inputted. In such a case, the intensity difference value distribution α is calculated from the image data of three types.

Considered hereinbelow are the brightness value C1(x1, y1, z1) at the coordinates (x1, y1, z1) of image data obtained with the first frequency filter, the brightness value C2(x1, y1, z1) at the coordinates (x1, y1, z1) of image data obtained with the second frequency filter, and the brightness value C3(x1, y1, z1) at the coordinates (x1, y1, z1) of image data obtained with the third frequency filter.

For example, when the frequency band from the structure which is wished to be enhanced is strong in the band of the second frequency filter and weak in the bands of the first and third frequency filters, the intensity difference value distribution information α is determined by the following Formula (5).

α(x1,y1,z1)=√((C2(x1,y1,z1)/C1(x1,y1,z1))̂2+(C2(x1,y1,z1/C3(x1,y1,z1))̂2) Formula (5)

As a result, the intensity difference value distribution information α in which the structure of a specific thickness is enhanced can be obtained. Further, by using the filters of three types, it is possible to enhance the structure with a specific thickness with an accuracy further increased with respect to that when the filters of two types are used.

The same effect is also obtained with the calculation method represented by formula (6) below.

α(x1,y1,z1)=C2(x1,y1,z1)/√(C1(x1,y1,z1)̂2+C3(x1,y1,z1)̂2) Formula (6)

It is also possible to calculate correlation coefficients between the intensities of the brightness value C1(x1, y1, z1), brightness value C2(x1, y1, z1), and brightness value C3(x1, y1, z1) and three intensities in the band of the first frequency filter, the band of the second frequency filter, and the band of the third frequency filter of the photoacoustic wave from the structure which is wished to be enhanced, and to use the correlation coefficients as the intensity difference value distribution information α. Such a calculation method also makes it possible to enhance accurately the structure with a specific thickness.

The schematic spectra which are depicted in FIG. 3 and based on the blood vessels of different thickness illustrate an example in which the frequency with a 0 intensity is not present. Therefore, in the division processing in the data value comparison unit 10, the denominator is not 0. However, the spectra of blood vessels which are actually obtained can have a frequency band in which the signal intensity is 0 or a very small value close to 0. In this case, the quotient obtained, that is, the ratio of signal intensities diverges. Accordingly, when the frequency band designated by the user is a range in which the intensity in the spectrum is 0 or a very small value close to 0, the effective method is to prevent the divergence of the signal intensity ratio by performing calculations using data outside the range in which the spectrum intensity is 0 or by adding a correction value to the denominator. A method for correcting the signal intensity ratio is not limited to that described hereinabove.

Further, the above-described embodiments can be considered not only as the object information acquiring apparatus and object information acquisition method, but also as a method for displaying an image relating to an object. The method for displaying an image relating to an object according to the present disclosure includes: (a) a step of displaying a first photoacoustic image relating to a group of blood vessels in an object; and (b) a step of forming a second photoacoustic image. The second photoacoustic image is formed by performing image processing on a first blood vessel contained in the group of blood vessels and a second blood vessel which differs in thickness from the first blood vessel, the image processing performed on the first blood vessel being different from that performed on the second blood vessel. The image processing is performed such that the visibility of the first blood vessel with respect to the second blood vessel in the first photoacoustic image is different from the visibility of the first blood vessel with respect to the second blood vessel in the second photoacoustic image.

The method for displaying an image relating to an object can be implemented by an image display device. The image display device can be configured by including functions of at least one component from among the filter circuit 8, image reconstruction unit 9, data value comparison unit 10, enhanced image creating circuit 11, image display system 12, and system control unit 6 depicted in FIG. 1.

The method for displaying an image relating to an object can be considered such that where the first blood vessel is thinner than the second blood vessel, the visibility of the first blood vessel with respect to the second blood vessel becomes higher in the second image than in the first image.

Further, as a specific method for realizing the image processing, the first photoacoustic image is formed using a time series signal obtained by receiving photoacoustic waves generated from the object due to irradiation of the object with light. Further, the visibility of the second blood vessel with respect to the first blood vessel in the second photoacoustic image can be made higher than the visibility of the second blood vessel with respect to the first blood vessel in the first photoacoustic image by using first image data obtained using a component of a first frequency band included in the time series signal and second image data obtained using a component of a second frequency band which is different from the first frequency band.

A configuration may be used in which the first and second blood vessels contained in the first photoacoustic image can be designated by the operator of the image display device. The image display device may be further provided with an input unit, and the first and second blood vessels may be designated by the designation received via the input unit. The operator can designate a blood vessel for which the visibility is wished to be changed in the first photoacoustic image displayed by the display system 12, while referring to the image. The first and second blood vessels may be individually designated by the operator, and where the operator designates a random region in the first ultrasound image, the image display device may specify blood vessels of mutually different thicknesses that are included in the designated region and notify the operator of those blood vessels prior to executing the image processing. When the operator wishes to take a blood vessel other than the first and second blood vessels, which have been notified by the image display device, as the first or second blood vessel, a configuration may be used in which the operator can further designate the first or second blood vessel. This designation may be the designation of the first and second blood vessels themselves or the designation of a region (region of interest) defined by a rectangle, a circle, an ellipse, or a polygon. It is desirable that the size of the region could be changed.

Further, the visibility can be changed by at least one of a brightness value, a contrast, and a hue in the first and second photoacoustic image.

### Other Embodiment

The present invention can be implemented also by a computer (or a device such as CPU and MPU) of a system or device that realizes the functions of the above-described embodiment by reading and executing a program recorded in a storage device. Further, the present invention can be also implemented by a method including the steps executed by a computer of a system or device that realizes the functions of the above-described embodiment by reading and executing a program recorded in a storage device. For this purpose, the program is provided to the computer, for example, via a network or from a recording medium of a type that can be used by the storage device (in other words, a computer-readable recording medium that non-temporarily holds data). Therefore, the computer, the method, the program and the computer-readable recording medium that non-temporarily holds the program are all also included in the scope of the present invention. the computer includes a device such as CPU and MPU. The program includes a program code and a program product.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-042732, filed on Mar. 4, 2015, and, Japanese Patent Application No. 2016-039289, filed on Mar. 1, 2016, which are hereby incorporated by reference herein in their entirety.

1. An object information acquiring apparatus comprising:
an extraction processing unit that extracts signal components of mutually different first and second frequency bands from an electric signal based on an acoustic wave propagating from an object caused by irradiation of the object with light;
an image signal generating unit that generates a first image signal based on the signal component of the first frequency band extracted by the extraction processing unit, a second image signal based on the signal component of the second frequency band extracted by the extraction processing unit, and a third image signal based on the electric signal; and
a weighting processing unit that performs weighting of a signal intensity of the third image signal on the basis of signal intensities of the first and second image signals.

2. The object information acquiring apparatus according to claim 1,
wherein the weighting processing unit generates a ratio of signal intensities of the first and second image signals.

3. The object information acquiring apparatus according to claim 2, wherein the weighting processing unit corrects the ratio of signal intensities when the ratio of signal intensities of the first and second image signals diverges.

4. The object information acquiring apparatus according to claim 3, wherein the weighting processing unit calculates the ratio of signal intensities after adding a correction value to the signal intensity, which is the denominator among the signal intensities of the first and second image signals.

5. The object information acquiring apparatus claim 2, wherein the weighting processing unit multiplies the signal intensity of the third image signal by the ratio.

6. The object information acquiring apparatus claim 1, wherein the third image signal is different from the first and second image signals.

7. The object information acquiring apparatus according to claim 6, wherein the third image signal is based on a signal component of a third frequency band which has been extracted by the extraction processing unit from the electric signal.

8. The object information acquiring apparatus according to claim 6, wherein the third image signal is obtained without processing the electric signal in the extraction processing unit.

9. The object information acquiring apparatus according to claim 1, wherein the third image signal is the first or second image signal.

10. The object information acquiring apparatus according to claim 1, wherein the image signal generating unit generates the third image signal on the basis of a signal component of a frequency band including the first and second frequency bands of the electric signal.

11. The object information acquiring apparatus according to claim 1, wherein at least part of the first frequency band overlaps part of the second frequency band.

12. The object information acquiring apparatus according to claim 1, wherein the first frequency band does not overlap the second frequency band.

13. The object information acquiring apparatus according to claim 1, wherein the extraction processing unit performs the extraction such that the intensity of the signal component of the first frequency band decreases as a distance from the central frequency of the first frequency band gradually increases and also such that the intensity of the signal component of the second frequency band decreases as a distance from the central frequency of the second frequency band increases gradually.

14. The object information acquiring apparatus according to claim 1, wherein the extraction processing unit extracts the intensity of the signal component of the first frequency band uniformly over the entire first frequency band and extracts the intensity of the signal component of the second frequency band uniformly over the entire second frequency band.

15. The object information acquiring apparatus according to claim 14, wherein the extraction processing unit extracts the signal component of the first frequency band by cutting off signal components of frequency bands other than the first frequency band and extracts the signal component of the second frequency band by cutting off signal components of frequency bands other than the second frequency band.

16. The object information acquiring apparatus according to claim 15, wherein the extraction processing unit has a low-pass filter that extracts a signal component of the frequency band with a smaller central frequency among the signal components of the first and second frequency bands, and a high-pass filter that extracts a signal component of the frequency band with a larger central frequency among the signal components of the first and second frequency bands.

17. An object information acquiring apparatus comprising:
an extraction processing unit that extracts a signal component of a first frequency band from an electric signal based on an acoustic wave propagating from an object due to irradiation of the object with light;
an image signal generating unit that generates a first image signal based on the signal component of the first frequency band extracted by the extraction processing unit, a second image signal obtained on the basis of the electric signal, without using the extraction processing unit, and a third image signal based on the electric signal; and
a weighting processing unit that performs weighting of a signal intensity of the third image signal on the basis of signal intensities of the first and second image signals.

18. The object information acquiring apparatus according to claim 17, wherein the weighting processing unit generates a ratio of the first and second image signals.

19. The object information acquiring apparatus according to claim 18, wherein the weighting processing unit multiplies the third image signal by the ratio.

20. The object information acquiring apparatus according to claim 17, wherein the third image signal is based on a signal component of a third frequency band which has been extracted by the extraction processing unit from the electric signal.

21. The object information acquiring apparatus according to claim 17, wherein the third frequency band is the first frequency band.

22. The object information acquiring apparatus according to claim 17, wherein the third image signal is obtained without processing the electric signal in the extraction processing unit.

23. The object information acquiring apparatus according to claim 1, further comprising:
an input unit for inputting the first and second frequency bands.

24. The object information acquiring apparatus according to claim 23, wherein a thickness of a structure inside the object, which is to be enhanced, is displayed on a display unit by the first and second frequency bands inputted through the input unit.

25. The object information acquiring apparatus according to claim 1, further comprising:
a receiving unit that receives an acoustic wave propagating from the object and generates the electric signal.

26. The object information acquiring apparatus according to claim 1, further comprising:
an irradiation unit for irradiating the object with light.

27. A method for displaying an image relating to an object, comprising:
a step of displaying a first photoacoustic image relating to a group of blood vessels in the object; and
a step of performing image processing on a first blood vessel and a second blood vessel differing in thickness from the first blood vessel, which are contained in the group of blood vessels, the image processing to be performed on the first blood vessel being different from that performed on the second blood vessel whereby a second photoacoustic image is formed,
wherein the image processing is performed such that a visibility of the first blood vessel with respect to the second blood vessel in the first photoacoustic image is different from a visibility of the first blood vessel with respect to the second blood vessel in the second photoacoustic image.

28. The method for displaying an image relating to an object according to claim 27, wherein
the first blood vessel is thinner than the second blood vessel, and
the image processing is performed such that the visibility of the first blood vessel with respect to the second blood vessel in the second photoacoustic image becomes higher than the visibility of the first blood vessel with respect to the second blood vessel in the first photoacoustic image.

29. The method for displaying an image relating to an object according to claim 27, wherein
the first photoacoustic image is formed using a time series signal obtained by receiving acoustic waves generated from the object due to irradiation of the object with light, and
the image processing is performed by using first image data obtained using a component of a first frequency band included in the time series signal and second image data obtained using a component of a second frequency band, which is different from the first frequency band, such that the visibility of the second blood vessel with respect to the first blood vessel in the second photoacoustic image becomes higher than the visibility of the second blood vessel with respect to the first blood vessel in the first photoacoustic image.

30. The method for displaying an image relating to an object according to claim 27, further comprising:
a step of receiving a designation of the first and second blood vessels contained in the first photoacoustic image.

31. The method for displaying an image relating to an object according to claim 27, further comprising:
a step of receiving a designation of a region including at least part of the first photoacoustic image which has been displayed,
wherein the image processing is performed on the first and second blood vessels included in the designated region.

32. The method for displaying an image relating to an object according to claim 30, further comprising:
a step of reporting on the first and second blood vessels included in the designated region, before the image processing.

33. The method for displaying an image relating to an object according to claim 27, wherein the visibility is at least one of a brightness value, a contrast, and a hue.
